(19) United States
(12) Patent Application Publication
(10) Pub. No.: US 2022/0105119 A1
(43) Pub. Date: Apr. 7, 2022

(54) DELIVERY OF AEROSOLIZED MICROMOLAR COMPOSITION CONCENTRATIONS

(71) Applicant: Amcyte Pharma Inc., Cambridge, MA (US)

(72) Inventor: Julio César Vega, Buenos Aires (AR)

(21) Appl. No.: 17/330,485

(22) Filed: May 26, 2021

Related U.S. Application Data
(60) Provisional application No. 63/034,705, filed on Jun. 4, 2020.

(51) Int. Cl. A61K 31/7048; A61K 9/12; A61K 9/00; A61K 47/12; A61K 47/10; A61K 47/06

(52) U.S. Cl. CPC A61K 31/7048; A61K 9/124; A61K 47/06; A61K 47/12; A61K 47/10; A61K 9/008

(57) ABSTRACT

Formulations and pressurized devices able to get Ivermectin concentrations in the micromolar range in the upper and lower respiratory tract and the oropharyngeal cavity are disclosed. The formulations and devices are used for the prevention or treatment of respiratory diseases caused by SARS CoV 2, such as pandemic COVID 19, and other viruses for which Ivermectin exerts viral inactivation at this concentration range.

### CROSS-REFERENCE TO RELATED APPLICATIONS

This application claims priority to, and the benefit under 35 U.S.C. § 119(e) of U.S. Provisional Application No. 63/034,705, filed Jun. 4, 2020. The entire contents of which are incorporated herein by reference in their entirety.

### TECHNICAL FIELD

The present disclosure relates to a pressurized device useful to get Ivermectin concentrations in the micromolar range in the upper and lower respiratory tract and the oropharyngeal cavity for the prevention or treatment of respiratory diseases caused by SARS CoV 2, such as pandemic COVID 19, and other viruses for which Ivermectin exerts viral inactivation at this concentration range.

### BACKGROUND

Coronaviruses are enveloped, positive-sense single-stranded RNA viruses. They have the largest genomes (26-32 kb) among known RNA viruses, and are phylogenetically divided into four genera (α, β, γ, δ), with beta coronaviruses further subdivided into four lineages (A, B, C, D). Coronaviruses infect a wide range of avian and mammalian species, including humans. Of the six known human coronaviruses, four of them (HCoV-OC43, HCoV-229E, HCoV-HKU1 and HCoV-NL63) circulate annually in humans and generally cause mild respiratory diseases, although severity can be greater in infants, elderly, and the immunocompromised. In contrast, the Middle East respiratory syndrome coronavirus (MERS-CoV) and the severe acute respiratory syndrome coronavirus (SARS-CoV), belonging to beta coronavirus lineages C and B, respectively, are highly pathogenic. Both viruses emerged into the human population from animal reservoirs within the last 15 years and caused outbreaks with high case-fatality rates.

SARS CoV 2 is the virus responsible for COVID 19, the pandemic disease initiated in Wuhan, China. It provokes severe acute respiratory syndromes, that may lead to death (Yang et al., Cellular & Molecular Immunology; doi.org/10.1038/s41423-020-0407-x). The high pathogenicity and airborne transmissibility of SARS-CoV and MERS-CoV, the high case-fatality rate, vaguely defined epidemiology, and absence of prophylactic or therapeutic measures against coronaviruses have created an urgent need for an effective vaccine and related therapeutic agents.

### SUMMARY

There is an urgent unmet need to provide aN easy-to-produce medicines to prevent and treat diseases caused by SARS CoV 2, for example, COVID-19, which would help avoid severe, probably fatal, respiratory syndromes. The disclosure surprisingly provides for compositions and methods of administering ivermectin in a formulation packaged into cans fitted with metering valves and suitable actuators to deliver the drug to the lower airways, nasal cavity, or oropharyngeal region. This device is useful to prevent or treat diseases caused by SARS-CoV2 such as COVID-19, i.e. the present pandemic disease. The invention can also be applied to the treatment of other viral diseases affecting the upper and/or lower respiratory tract and/or the oropharyngeal region, such as influenza.

Accordingly, in these embodiments, suitable pharmaceutical formulations and devices are provided.

In embodiments an amount of ivermectin is administered to the site of action (nasal cavity, nasopharynx, lower airways and/or oropharyngeal region), so that the final concentration achieved is above 2 μM (equivalent to 1.7 μg/mL) or even higher than 5 μM (equivalent to 4.4 μg/mL).

In certain embodiments the formulation comprises a solution of ivermectin in a s pharmaceutically acceptable solvent and a pharmaceutically acceptable propellant. The pharmaceutically acceptable solvent comprises: anhydrous ethanol, 96° ethanol, isopropanol, propylene glycol, glycerin. In certain embodiments, more than one of these solvents can be used in the formulation. Pharmaceutically acceptable propellants comprise: propane, n-butane, isopropane, isopentane, n-pentane, Norflurane (HFA 134a), Apaflurane (HFA 227 ea). In certain embodiments, the solution contains other excipients such as tartaric acid, citric acid, hydrochloric acid, oleic acid, sorbitan trioleate, lecithin and other used in inhalation delivery. The said solution is packaged into cans and a metering valve is crimped onto it. Valvesy have metered volumes from 10 to 200 μL per shot.

In certain embodiments, the actuator is designed for inverted or upright use. In the latter case the valve is fitted with a dip tube submerged into the liquid phase of the pressurized composition. In case of inverted-use actuators, the unit is placed in valve-down position and pressed downwards, so that the formulation present in the metering chamber of the valve is released through an orifice in the actuator into the air as a mist of droplets driven forward by the pressure caused by the flash vaporization of the propellant when leaving the metering chamber. This mist is delivered to the nose, the oropharyngeal cavity, or the lower airways by means of different actuators. In case of upright-use actuators, when the actuator is pressed downwards, the formulation in the metering chamber of the valve is released through an orifice in the actuator into the air as a mist of droplets driven forward by the pressure caused by the flash vaporization of the propellant when leaving the metering chamber. This mist is delivered to the nasal cavity (nose), or the oropharyngeal cavity.

In certain embodiments, a suitable oral actuator with an orifice diameter not less than 0.50 mm is used to deliver ivermectin to the oropharyngeal cavity. This route provides protection against contagion of SARS CoV 2 disease by lowering the viral load of saliva of infected people.

In another embodiment, a suitable nasal actuator with an orifice diameter not less than 0.50 mm is used to deliver ivermectin to the nasal cavity. This route provides protection against contagion of SARS CoV 2 disease to people in close and frequent contact with infected people, such as medical staff or people living with them.

In another embodiment, a suitable nasal actuator with an orifice diameter less than 0.50 mm is used to deliver Ivermectin to the lower airways by inhalation. This route provides effective concentrations of Ivermectin in the lower airways to reduce the viral load in patients at a certain stage in the disease.

Other aspects are discussed infra.

### Definitions

The terminology used herein is for the purpose of describing particular embodiments only and is not intended to be limiting of the invention. Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered.

As used in this specification and the appended claims, the term “or” is generally employed in its sense including “and/or” unless the content clearly dictates otherwise.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

Ranges: throughout this disclosure, various aspects of the invention can be presented in a range format. The description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

FIG. 1 shows the impurity profile of the lot of drug substance used to manufacture the formulations was compared with the values obtained after 1 week at 50° C. (an extremely high stress condition for an MDI).

### DETAILED DESCRIPTION

The present disclosure is based on the surprising discovery that formulations of dissolved ivermectin using pharmaceutically acceptable ingredients are physically and chemically stable and can administer a preventive and therapeutically effective amount of this drug substance to the sites where SARS CoV 2 is mostly located by means of pharmaceutically acceptable devices. All these formulations and devices deliver ivermectin to reach a therapeutically relevant final concentration in the external liquid of each administration route by one or more actuations in order to treat viral infections caused by SARS-CoV-2 or other viruses.

Ivermectin inhibit SARS CoV 2 replication in Vero cells culture with an IC₅₀ of 2 μM, corresponding to a mass concentration of about 1.7 μg/Ml. IC₅₀ is the concentration of Ivermectin capable of achieving 50% viral inhibition) and achieving ca. 5,000 times viral RNA reduction at a concentration of 5 M (equivalent to ca. 4.4 μg/mL). However, this concentration does not seem achievable in the airways or in the oropharyngeal region with the existing pharmaceutical dosage forms and administration routes (topical, oral, injection). Moreover, ivermectin inhibits the replication of other viruses, as well. Therefore, there is an urgent need to provide a drug product able to achieve concentrations of ivermectin in the upper and lower respiratory tract and oropharyngeal cavity at this level and above.

It is useful to treat other diseases caused by viruses for which ivermectin exerts antiviral activity in the concentration range of 0.003-10 μM.

Ivermectin is a well-known antiparasitic semisynthetic drug approved in the US as:

A 3-mg oral tablet indicated for the treatment of intestinal (i.e., nondisseminated) strongyloidiasis due to the nematode parasite Strongyloides stercoralis and the treatment of of onchocerciasis due to the nematode parasite Onchocerca volvulus.
A 0.5% w/w lotion indicated for topical treatment of head lice infestations.
A 1% w/w topical cream indicated for the treatment of inflammatory lesions of rosacea.
There are 6—mg tablets and 0.6% oral drops available in other countries like Argentina, as well. They are all antiparasitic drugs indicated for scabies in addition to the indications approved by FDA in the US.

Ivermectin consists of a mixture of two chemical compounds indicated as Formula I below.

H₂B₁ₐ (CAS 70161-11-14) and H₂B_{1b} (CAS No. 70288-86-7). Both USP (United States Pharmacopoeia) and Ph Eur (European Pharmacopoeia) establish that the ratio the areas by liquid chromatography H₂B₁ₐ/(H₂B₁ₐ+H₂B_{1b}) should be not less than 90.0%.

| | | | Formula I |
|---|---|---|---|
| 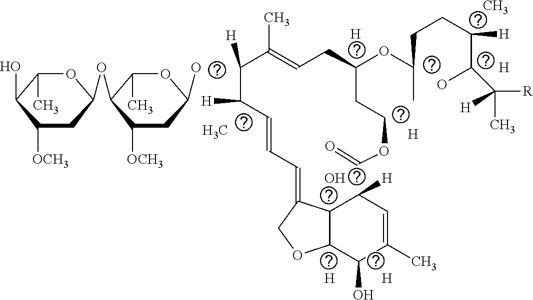 | | | |
| Component | R | Molecular formula | [symbol] |
| [symbol] | [symbol] | [symbol] | 875 |
| [symbol] | [symbol] | [symbol] | 861 |

Ivermectin B1a: [70161-11-4]
Ivermectin B1b: [70288-86-7] [symbol]
indicates data missing or illegible when filed

This drug substance has been found to exert in vitro viral replication inhibition at concentrations between 3 nM to 10 μM on several viruses, such as flaviviruses (yellow fever virus, West Nile virus, dengue virus, Japanese encephalitis virus, tick-borne encephalitis virus) via inhibition of NS helicase enzymatic activity (Mastrangelo 2012, Crump 2017). It has been recently found to inhibit replication of SARS CoV 2 in vitro in Vero cells culture (Caly 2020). Its activity against several RNA viruses such as the SARS-CoV-2 may be related to mechanisms that inhibit importin α/β-mediated nuclear transport (Caly 2020). In the particular case of SARS CoV 2, IC₅₀ (i.e. the concentration of Ivermectin needed to inhibit 50% virus replication) is ca. 2 μM and it achieves 5,000-fold reduction in viral RNA in reference to placebo at 5 μM (Caly 2020) 48 hours after one single application. This corresponds to mass concentrations of ca. 1.7 μg/mL (2 μM) and 4.4 μg/mL (5 μM), respectively (It is considered herein that the weighted average weight of Ivermectin=0.9×875+0.1×861=873.6). In conclusion, a single application of Ivermectin at a concentration of 4.4 μg/mL can inhibit viral replication of SARS-CoV-2 almost completely after 48 hrs.

On the other hand, plasma concentrations achievable via oral administration are much lower than the concentration needed to achieve in vitro viral replication inhibition as taught by several authors after a single dose of ivermectin, some of them well above the recommended dose for marketed products:

Maximum Plasma Concentrations (ng/mL) achieved
after single dose oral Ivermectin (tablets)

| | | Guzzo | Baraka | Schulz | Schulz |
|---|---|---|---|---|---|
| Doses | | 2002 | 1996 | 2019 | 2018 |
| 150 | μg/kg (fasted) | — | aprox. 55 | — | — |
| 600 | μg/kg (children, fed) | — | — | 66.1 | — |
| 200 | μg/kg (fed) | — | — | — | 40.1 |
| 30 | mg (fed) | 260.5 | — | — | — |
| 30 | mg (fasted) | 84.8-84.7 | — | — | — |
| 60 | mg (fasted) | 165.2-186.2 | — | — | — |
| 90 | mg (fasted) | 158.1 | — | — | — |
| 120 | mg (fasted) | 247.8 | — | — | — |

“fasted” means that Ivermectin was ingested after fasting overnight.
“fed” means that Ivermectin was ingested after breakfast. Breakfast consisted of oily fish and bread in the studies carried out by Schulz. In the study performed by Guzzo it consisted of a standard high-fat breakfast (31.3 g protein, 57.16 g carbohydrates, 48.6 g fat, 784 kcal) Time elapsed to reach maximum concentration is ca. 4-5 hours after ingestion in adults and 6-7 hours in children.

As it can be seen in the table above, the highest found maximum plasma concentration is 186.2 ng/mL which is ca. 24 times less than the inhibitory concentrations (Caly 2020). Moreover, most Ivermectin (93%) is bound by plasma proteins (Thomas 2020), i.e. free Ivermectin concentration is just 7/100 of the total plasma concentration. However, there are preliminary results of oral administration of Ivermectin in doses of ca. 150 μg/kg that may have played a role in decreasing the mortality rate of COVID 19 patients, particularly for patients submitted to mechanical ventilation as reported in literature (Thomas 2020). This may be partially due to the contribution of other standard-of-care treatments and should be tested in randomized clinical trials for certainty. It has been hypothesized that accumulation of Ivermectin in the lung tissue could explain this behavior although lung concentration is estimated to be just 2.2 times that of plasma (Thomas 2020), like what has been observed previously in animals (Lifschitz 2000). However, accumulation does not explain completely the potential efficacy of oral ivermectin, particularly in doses of 150 μg/kg as reported in literature (Thomas 2020), because the highest plasma concentration would be 2.2×186.2 ng/mL≈410 ng/mL, i.e. more than ten times lower than the amount needed to inhibit the virus action in vitro.

Regarding safety, ivermectin has been used for several years in the US and other countries to treat parasites. as reported before. in single oral doses up to 15 mg or ca. 200 μg/kg. In the case of endemic lymphatic filariasis, even single annual doses of 300-400 μg/kg has been used (Guzzo 2002). Safety of oral ivermectin is not considered a concern in the doses use today. However, doses will probably prove insufficient for SARS CoV 2 treatment and other viruses based on pharmacokinetic results available so far and the in vitro results found by Caly (Caly 2020). Considering the highest Cₘₐₓ achieved so far and the factor 2.2 to estimate the concentration in the lungs, the highest concentration achievable there would be 2.2×260.5 ng/mL=573.1 ng/mL≈7.6 times less than required for inhibition of SARS CoV 2 replication estimated as 4.4 μg/mL). The same is estimated to hold for nasal and oropharyngeal cavities.

In the case of the nasal cavity, considering that the surface area of the nasal mucosa is 100-250 cm² (Bitter 2011, Garcia 2009, Gizurarson 2012, Pires 2009) and that the airway surface liquid height, though variable, is 5-15 μm (Helassa 2014, Wagenman 1992) the airway surface liquid volume is within 50 and 375 μL. That means that administering 100 μg of Ivermectin per nostril in a 50-μL shot, would achieve a concentration of 533 μg/mL, well above what is needed to completely inactivate the virus. Systemically, there would be no safety concern, because the dose per kg body weight would be 200 μg/70 kg=2.9 μg/kg (taking 70 kg as average body weight for an adult), i.e., whereas the oral dose is 150 μg/kg, equivalent to ca 52 times the proposed nasal dose. Lower doses would be achievable as well by using a smaller metered volume or more dilute solutions.

In case of oropharyngeal region, the amount of saliva is estimated as 0.8 mL (Humphrey 2001). One 50-μL shot containing 100 μg of Ivermectin would achieve an immediate concentration of 125 μg/mL, i.e. much higher than needed to get viral inactivation (IC₅₀=1.7 μg/mL).

Inhalation has already been recently signaled as a more appropriate route to administer ivermectin (Thomas 2020, Chaccour 2020, Errecalde 2020). Considering experimental measurements by several authors, the amount of airway surface liquid in the lower airways (trachea, bronchii, bronchioli and alveoli) is between 10 and 70 mL (Frolich 2016). This means that delivering 308 μg of ivermectin into the lower airways would be enough to get a drug concentration of 4.4 μg/mL considering a 70-ml airway surface liquid volume To deliver this amount of ivermectin it is needed to be considered that solution pressurized metered dose inhalers usually have a deposition of ca. 35% in the lower airways. If 308 μg of ivermectin is to be delivered to the lower airways, a dose of at least 0.88 mg needs to be orally inhaled. This delivered dose or a higher one is easily reached by administering one or more shots of the present invention as a metered dose inhaler. As examples, this or larger amounts of drug is delivered to the lungs either by inhaling one shot containing not less than 880 μg, or 2 shots containing not less than 440 μg, or 4 shots containing at least 220 μg per puff.

Regarding systemic exposure, the invention herein is certainly advantageous compared to the oral route. Let us take a dose of 0.88 mg of inhaled ivermectin. Part of this dose (ca. 65%, 0.57 mg) would be ingested and the remaining part (ca. 35%, i.e. 31 mg) would be deposited in the lower airways. Oral bioavailability of ivermectin is approximately 40-50% (Therapeutic Goods Administration, Australian Public Assessment Report for Ivermectin, 2015). If we consider that the fraction of ivermectin deposited in the lower airways is 100% bioavailable (worst-case scenario), the systemic exposure of this dose would be 0.57×0.4+0.31=0.54 mg/70 kg for a 70-kg adult, i.e. 7.7 μg/kg. The approved oral dose of ivermectin is up to 200 μg/kg, considering a 40% bioavailability, the systemic exposure would be 80 μg/kg, i.e. more than 10 times the systemic exposure estimated for this dose of inhaled ivermectin.

However, formulation of ivermectin in solution is challenging, because of its poor water solubility and quick oxidation. As known in the art, ivermectin needs to be stored between 2 and 8° C. if no antioxidant is added (USP, Ph Eur monographs). Even sophisticated formulations, such as nanosuspensions (Starkloff 2016) and cyclodextrin complexes (Astier 2015), have been disclosed in the art to overcome poor ivermectin solubility and chemical stability. Moreover, forced degradation studies have shown that ivermectin is susceptible to oxidation and hydrolysis (Ali 2011). In summary, ivermectin remains a difficult drug substance to formulate and stabilize in a solution suitable for inhalation, nasal and oropharyngeal administration.

Surprisingly, it was discovered herein that a formulation containing ethanol and non-ozone depleting propellants suitable for nasal, oropharyngeal and inhalation administration of ivermectin has been found to be physically and chemically stable and able to deliver an amount of ivermectin allowing to achieve therapeutically effective concentrations in the micromolar range needed to inhibit several viruses, including SARS CoV 2. In some embodiments, the formulations free from antioxidants are stable even at temperatures of 40° C. (at least 6 months) and 50° C. (at least 1 month).

In certain embodiments an amount of ivermectin is administered to the site of action, so that the final concentration achieved is above 5 μM, i.e. a concentration that inhibits SARS CoV 2 and other viruses.

In certain embodiments a solution of Ivermectin in a suitable mixture of pharmaceutically acceptable solvent and a pharmaceutically acceptable propellant. Pharmaceutically acceptable solvent is taken from the group: anhydrous ethanol, 96° ethanol, isopropanol, propylene glycol, glycerin. In certain embodiments, more than one of these solvents are used in the formulation. Pharmaceutically acceptable propellant is taken from the group: propane, n-butane, isopropane, isopentane, n-pentane, Norflurane (HFA 134a), Apaflurane (HFA 227 ea). Other pharmaceutically acceptable propellants can be used. The said solution is packaged into cans fitted with a metering valve and an actuator. Valves may have metered volumes from 10 to 200 μL per shot. Upright—use and inverted—use actuators can be utilized. The orifice diameter should lie between 0.2-1 mm. Upright-use actuators require the valve to be fitted with a dip tube immersed into the liquid phase of the pressurized composition.

In case of upright-use actuators, when the actuator is pressed downwards, the formulation in the metering chamber of the valve is released through an orifice in the actuator into the air as a mist of droplets driven forward by the pressure caused by the flash vaporization of the propellant when leaving the metering chamber. This mist is delivered to the nasal cavity (nose), or the oropharyngeal cavity.

In case of inverted-use actuators, the unit is inverted (valve-down position) and when the can is pressed downwards, the formulation in the metering chamber of the valve is released through an orifice in the actuator into the air as a mist of droplets driven forward by the pressure caused by the flash vaporization of the propellant when leaving the metering chamber. In certain embodiments, this mist is delivered to the nose, the oropharyngeal cavity, or the lower airways by means of different actuators.

In some embodiments the said solution may contain other excipients such as tartaric acid, citric acid, hydrochloric acid, oleic acid, sorbitan trioleate, lecithin and other used in inhalation delivery.

In some embodiments, the said solution comprises excipients such as butylated hydroxytoluene and butylated hydroxyanisole.

In certain embodiments, a suitable oral actuator with an orifice diameter between 0.20-1 mm may be used to deliver ivermectin to the oropharyngeal cavity. This route provides protection against transmission of SARS CoV 2 or other viral diseases by lowering the viral load of saliva of infected people. Oral actuators are generally used in the inverted position.

In another embodiment, a suitable nasal actuator with an orifice diameter between 0.2 and 1 mm is used to deliver Ivermectin to the nasal cavity. This route provides protection against transmission of SARS CoV 2 and other viruses to subjects in close and frequent contact with infected people, such as medical staff or people living with ill persons. For nasal administration both upright—use and inverted—use actuators are suitable.

In another embodiment, a suitable oral actuator with an orifice diameter between 0.2 and 1 mm is used to deliver ivermectin to the lower airways by inhalation. This route provides adequate concentrations of ivermectin in the lower airways to reduce the viral load in patients at a certain stage in the disease.

### EXAMPLES

Examples are provided below to facilitate a more complete understanding of the invention. The following examples illustrate the exemplary modes of making and practicing the invention.

### Example 1

Preliminary tests of solubility of Ivermectin in Norflurane/Ethanol mixtures showed that 0,4% m/V required ca. 10% m/V ethanol to stay in solution after bringing to mass (100%) with Norflurane.

A new solubility test run was performed. Ivermectin was agitated with different portions of ethanol in pressurizable glass tubes. This ethanolic concentrate was stored at 2-8° C. overnight. In those tubes where no precipitation was observed, Norflurane (HFA134a) was added under pressure through a continuous valve fitted onto the tube. Tube was agitated and visually inspected for precipitation of Ivermectin after overnight storage at 2-8° C. The following table summarizes the results:

| | | | | After | | | After |
|---|---|---|---|---|---|---|---|
| | | | | overnight | | After | overnight |
| Test | Ivermectine | Ethanol | Dissolves | storage at | | adding | storage at |
| Number | % m/V | % m/m | in ethanol? | 2-8° C. | Norflurane | Norflurante | 2-8° C. |
| EN 04- | 0.4 | 10 | Yes | Clear | s.q. 100% | Clear | Clear |
| PR2/A-1 | | | | solution | | solution | solution |
| EN 05 | 0.8 | 10 | Yes | Precipitate | No further testing | | |
| EN 06 | 1.2 | 10 | No | No further testing | | | |
| EN 07 | 1.6 | 10 | No | No further testing | | | |
| EN 08- | 0.8 | 15 | Yes | Clear | s.q. 100% | Clear | Clear |
| PR3/A-1 | | | | solution | | solution | solution |
| EN 09- | 0.8 | 20 | Yes | Clear | s.q. 100% | Clear | Clear |
| PR4/A-1 | | | | solution | | solution | solution |
| EN 10- | 1.2 | 20 | Yes | Clear | s.q. 100% | Clear | Clear |
| PR5/A-1 | | | | solution | | solution | solution |
| EN 11- | 1.2 | 25 | Yes | Clear | s.q. 100% | Clear | Clear |
| PR6/A-1 | | | | solution | | solution | solution |
| EN 12- | 1.6 | 25 | Yes | Clear | s.q. 100% | Clear | Clear |
| PR7/A-1 | | | | solution | | solution | solution |
| EN 13- | 1.6 | 30 | Yes | Clear | s.q. 100% | Clear | Clear |
| PR8/A-1 | | | | solution | | solution | solution |

### Example 2

Based on the tests described in example 1, the following formulations were manufactured and tested:

| | Ivermectin | Ethanol | Norflurane | Dose | Density |
|---|---|---|---|---|---|
| Formulation | % m/V | % m/m | % m/m | μg/shot | (g/cm³) |
| A | 0.4 | 10 | s.q. 100 | 200 | 1,162 |
| B | 0.8 | 15 | s.q. 100 | 400 | 1,133 |
| C | 1.2 | 20 | s.q. 100 | 600 | 1,105 |
| D | 1.6 | 25 | s.q. 100 | 800 | 1,078 |

Reference: as it is usual in the design of pressurized metered dose inhaler formulations, density is calculated assuming no volume variation after mixing of ethanol and Norflurane.

The composition of each formulation per shot is as follows:

| | Mass of components per 50-μL shot (mg) | | | |
|---|---|---|---|---|
| Components | A | B | C | D |
| Ivermectin | 0.20 | 0.40 | 0.60 | 0.80 |
| Ethanol anhydrous | 5.8 | 8.5 | 11.0 | 13.5 |
| Norflurane (HFA 134a) | 52.1 | 47.7 | 43.6 | 39.6 |
| Shot mass | 58.1 | 56.6 | 55.2 | 53.9 |

To manufacture these formulations Ivermectin was dissolved in the corresponding amount of ethanol anhydrous and filled into aluminum alloy cut edge cans. 50-μL metering valves were crimped onto them and Norflurane was added under pressure through the valves.

These formulations were stored during 7 days at 50° C. and tested for related substances (to check chemical stability) and deposition of the emitted dose (to check the fraction of particles able to be inhaled delivered by each shot).

All formulations were assayed from the valve (metered dose) before and after storage with the following results expressed as percent of expected value (i.e. 200 μg, 400 μg, 600 μg and 800 μg per shot):

| | Initial (before | After 7 days at |
|---|---|---|
| Formulation | storage) | 50° C. |
| A | 99.9% | 105.2 |
| B | 96.9% | 100.0 |
| C | 97.3% | 97.9 |
| D | 91.7% | 101.7 |

The usual acceptance limits for an MDI are 80-120%. Therefore, the results are excellent regarding assay. The values after storage may be higher due to some leak of propellant due to this very high storage temperature, i.e. 50° C.

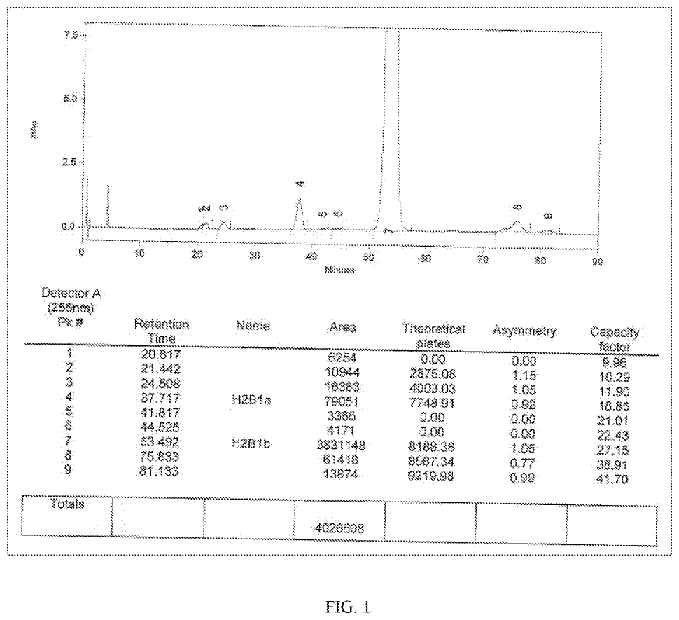

HPLC method used renders chromatograms like the one depicted in FIG. 1 for drug substance and drug product. Observing chromatogram in FIG. 1, it is seen that peaks 4 and 7 correspond to H₂B₁ₐ and H₂B_{1b} present in ivermectin, i.e. drug substance. The rest of the peaks may be attributed to related substances. Related substances are substances structurally similar to the drug substance. Some of them come from synthesis impurities and are not increased during shelf life because they cannot form from degradation of the drug substance. Others are degradation products.

Degradation studies were performed to attribute the rest of the related substances to different degradation pathways. Peak #6 (retention time=ca. 44.5 min, retention time relative to H2B1b (RRT)=0.83) is generated by oxidation with H₂O₂ (Procedure: 20 mg of Ivermectin was dissolved in 15 mL of Acetonitrile:Water 1:1 mixture and 0.3 mL of H₂O₂ 100 volumes was added and left at 50° C. during 1 hour. The reaction mixture was brought to 50 mL with Methanol and injected into the high-pressure liquid chromatograph), whereas peak #8 (retention time=ca. 75.8, relative retention time relative to H2B1b (RRT)=1.4 is generated by reaction in alkaline media (Procedure: 20 mg of Ivermectin were dissolved in 15 mL of Acetonitrile:Water 1:1 mixture and 2 mL of NaOH 1N were added and left at 50° C. during 1 hour. The reaction mixture was neutralized with HCl 1N, brought to 50 mL with Methanol and injected into the high-pressure liquid chromatograph)

FIG. 1 shows the impurity profile of the lot of drug substance used to manufacture the formulations was compared with the values obtained after 1 week at 50° C. (an extremely high stress condition for an MDI).

The results of related substances are as follows:

| | Related substances identified by | | | | | | |
|---|---|---|---|---|---|---|---|
| | their RRT (% total HPLC area) | | | | | | |
| Test item | 0.39 | 0.40 | 0.46 | 0.78 | 0.83 | 1.42 | 1.52 |
| Drug substance | 0.3 | 0.3 | 0.5 | 0.1 | ND | 2.9 | 0.7 |
| Formulation A | 0.3 | 0.4 | 0.6 | ND | ND | 2.4 | 0.7 |
| Formulation B | 0.3 | 0.5 | 0.6 | ND | ND | 2.6 | 0.6 |
| Formulation C | 0.3 | 0.4 | 0.7 | 0.1 | 0.2 | 2.5 | 0.6 |
| Formulation D | 0.3 | 0.4 | 0.5 | ND | ND | 2.2 | 0.5 |

As can be seen, even after 1 week at 50° C. no significant degradation is detected. Differences between drug substance and stored formulation may be explained by random analytical variance (ca. 0.2%) among HPLC runs. This is a surprising result considering its tendency towards oxidation, as previously described.

The packaged formulations fitted with an inverted-use actuator for oral inhalation having a 0.25-mm orifice diameter were tested in duplicate for deposition of the emitted dose (British Pharmacopoeia 2019, Appendix XII C—Consistency of Formulated Preparations—Preparations for Inhalation: Aerodynamic Assessment of Fine Particles—Apparatus A—Glass impinger) with the following results:

| Formulation | Fine particles mass (μg) | Fine particle fraction (%) |
|---|---|---|
| A | 92-102 | 46-51 |
| B | 200-204 | 50-51 |
| C | 222-240 | 37-40 |
| D | 240-264 | 30-33 |

Fine particle fraction decreases as ethanol content increases. A highly probable explanation is that ethanol stays unevaporated and contributes to the formation of larger droplets. All formulations have an acceptable deposition. To further determine aerodynamic particle size distribution, formulation D was analyzed using Andersen Cascade Impactor (British Pharmacopoeia 2019, Appendix XII C—Consistency of Formulated Preparations—Preparations for Inhalation: Aerodynamic Assessment of Fine Particles—Apparatus D—Andersen Cascade Impactor). Results are summarized as follows:

| Fine particle mass | | |
|---|---|---|
| (μg) | MMAD (μm) | GSD (μm) |
| 204.2 | 1.6 | 3.0 |

These results are like those of other solution MDIs and correlate well with in vivo inhaled mass in the lower airways (De Backer 2010). Formulations A and B have certainly higher fine particle masses and similar particle size distribution (MMAD and GSD). Considering that the fine particle fraction determined in the deposition of the emitted dose reaches the lower airways, Formulation A delivers ca. 100 μg ivermectin in the lower airways per shot (fine particle mass per shot) and formulation B delivers ca. 200 μg per shot. Four shots of formulation A or two shots or formulation B reach local concentrations of ivermectin in the range 40-5.6 μg/mL in the lower airways (taking 70 and 10 mL, respectively, as total airway surface liquid estimation for the lower airways). This means that four shots of formulation A or two shots of formulation B suffice to reach a concentration higher than 4.4 μg/mL of ivermectin in the lower airways that would practically eliminate SARS CoV 2 and several other viruses with similar or lower IC₅₀ or higher (400 μg inhaled ivermectin divided by 10 mL would render a concentration of 40 μg/mL and divided by 70 mL would render a concentration of 5.7 μg/mL in the lower airways).

These formulations are easy to manufacture, physically stable even at low temperatures (2-8° C.) and chemically stable even at extremely high temperatures (1 week at 50° C.). They are useful to reach the lower airways and get a suitable ivermectin local concentration to inactive viruses with IC₅₀ in the micromolar range or lower.

### Example 3

The compositions of example 2 were put on longer-term stability studies in particularly challenging conditions, such as climatic zone IVb (30° C./75% RH) and accelerated conditions (40° C./75% RH). Results are surprisingly within acceptable ranges after 6-month storage as can be seen in the following tables:

Results obtained after 6-month storage at climatic zone IVb (30° C./75% RH)

| Test | Acceptance | Formulation | Formulation | Formulation | Formulation |
|---|---|---|---|---|---|
| parameter | limits | A | B | C | D |
| Assay | 80-120% | 99.3% | 97.5% | 93.3% | 95.1 |
| H2B1a/ | ≥90% | 98.2% | 98.2% | 98.2% | 98.2 |
| (H2B1a + | | | | | |
| H2B1b) Ratio | | | | | |
| Uniformity | 75-125% | 106.3-120.7% | 97.5-104.1% | 99.0-101.3% | 96.5-104.0% |
| of dosage | See Note 1 | | | | |
| units | below | | | | |
| Related | RRT 1.3- | 1.2% | 1.2% | 1.1% | 1.1% |
| substances | 1.5, ≤3% | | | | |
| | Other | 0.4%, 0.2%, | 0.4%, 0.2%, | 0.4%, 0.2%, | 0.5%, 0.2%, |
| | impurities | 0.1%, 0.3%, | 0.1%, 0.1%, | 0.1%, 0.3%, | 0.1%, 0.3%, |
| | | 0.2% | 0.3%, 0.2% | 0.2% | 0.2% |
| | Total ≤6.0% | 2.3% | 2.3% | 2.3% | 2.4% |
| Deposition | Not less | 59% | 59% | 45% | 36% |
| of the | than 25% | | | | |
| emitted dose | | | | | |

Results obtained after 6-month storage at 40° C./75% RH (accelerated conditions)

| Test | Acceptance | Formulation | Formulation | Formulation | Formulation |
|---|---|---|---|---|---|
| parameter | limits | A | B | C | D |
| Assay | 80-120% | 99.4% | 94.9% | 96.8% | 91.1% |
| H2B1a/ | ≥90% | 98.2% | 98.3% | 98.3% | 98.2% |
| (H2B1a + | | | | | |
| H2B1b) Ratio | | | | | |
| Uniformity | 75-125% | 98.0-105.4% | 98.9-101.2% | 96.4-103.6% | 98.1-101.6 |
| of dosage | See Note 1 | | | | |
| units | below | | | | |
| Related | RRT 1.3- | 1.2% | 1.1% | 1.2% | 1.1% |
| substances | 1.5, ≤4% | | | | |
| | Other | 0.5%, 0.2%, | 0.5%, 0.2%, | 0.5%, 0.2%, | 0.5%, 0.2%, |
| | impurities | 0.2%, 0.1%, | 0.1%, 0.3%, | 0.1%, 0.3%, | 0.1%, 0.3%, |
| | | 0.2% | 0.2% | 0.2% | 0.1% |
| | Total ≤6.0% | 2.4% | 2.4% | 2.5% | 2.3% |
| Deposition | Not less | 66% | 59% | 40% | 34% |
| of the | than 25% | | | | |
| emitted dose | | | | | |

Furthermore, deposition of the emitted dose does not change significantly from initial values (see values in Example 2).

It was even found that formulation free from antioxidants are stable even at temperatures of 40° C. (at least 6 months) and 50° C. (at least 1 month).

### Example 4

Compositions are depicted in the following table:

| | Mass of components per 50-μL shot (mg) | | | |
|---|---|---|---|---|
| Components | A | B | C | D |
| Ivermectin | 0.20 | 0.40 | 0.60 | 0.80 |
| Ethanol anhydrous | 5.8 | 8.5 | 11.0 | 13.5 |
| Norflurane (HFA 134a) | 52.1 | 47.7 | 43.6 | 39.6 |
| Shot mass | 58.1 | 56.6 | 55.2 | 53.9 |

To manufacture these formulations ivermectin was dissolved in the corresponding amount of ethanol anhydrous and filled into aluminum alloy cut edge cans. 50-μL metering valves were crimped onto them and Norflurane was added under pressure through the valves.

When these package units are fitted with an oral actuator with an orifice diameter of 0.2-1 mm and they are actuated without inhaling, droplets remain in the pharynx and oral cavity, particularly if orifice diameters are 0.5 mm or larger. As ca. 0.8 mL of saliva are present in the oral cavity (Humphrey 2001), the amount of ivermectin deposited reaches an average concentration of ca. 220 μg/mL (considering that ca. 10% of the shot mass remains within the actuator). This concentration is high enough to allow for the significant viral load reduction of SARS CoV 2 (IC₅₀=1.7 μg/mL and practically complete elimination at 4.4 μg/mL) and other viruses from saliva and could help decrease the reproduction number in an epidemic or pandemic (i.e. the number of cases directly generated by one case in a population).

### Example 5

Compositions depicted in the following table, packaged in aluminum alloy cans, fitted with 100-μL valves and oral actuators:

| | Mass of components per 100-μL shot(mg) | | | |
|---|---|---|---|---|
| Components | A | B | C | D |
| Ivermectin | 0.40 | 0.80 | 1.20 | 1.60 |
| Ethanol anhydrous | 11.6 | 17.0 | 22.0 | 27.0 |
| Norflurane (HFA 134a) | 104.2 | 95.4 | 87.2 | 79.2 |
| Shot mass | 116.2 | 113.2 | 110.4 | 107.8 |

To manufacture these formulations ivermectin was dissolved in the corresponding amount of ethanol anhydrous and filled into aluminum alloy cut edge cans.

When these packaged units are with an inverted-use nasal actuator (These actuators have one orifice through with the formulation is sprayed and ends in two channels, one entering each nostril), one shot is divided between both nostrils. The volume of the airway surface liquid in the nose is estimated between 50 and 375 μL based on literature sources as previously discussed. Thus, one shot of formulation A, for example, delivers ivermectin reaching a local concentration between ca. 8000 μg/mL and 533 μg/mL. This concentration decreases because of mucociliary clearance but remains a certain time well above the concentration needed to inhibit replication of SARS CoV 2 and other viruses. This product helps nurses and physicians to decrease the probability of transmission when assisting a patient.

### Example 6

Formulations to administer 50 μg and 100 μs of ivermectin per shot are presented in the following table:

| | Mass of components per 50-μL shot (mg) | |
|---|---|---|
| Components | A | B |
| Ivermectin | 0.05 | 0.10 |
| Ethanol anhydrous | 5.81 | 5.81 |
| Norflurane (HFA 134a) | 52.25 | 52.20 |
| Total mass per shot | 58.11 | 58.11 |

To manufacture these formulations ivermectin is dissolved in ethanol anhydrous. Norflurane is added then under pressure in an airtight mixing vessel and the formulation is filled into the cans through the 50-μL valves already crimped onto them.

Oral actuators with orifice diameter between 0.2 and 1 mm are used for oropharyngeal deposition without inhaling. Oral actuators with orifice diameter not larger than 0.7 mm are used for lower airways deposition via inhalation. Nasal actuators are used for nasal administration.

### Example 7

Following formulations packaged in polyethylene terephthalate cans fitted with 50-μL valves and actuators for oral inhalation:

| | Mass of components per 50-μL shot (mg) | | | |
|---|---|---|---|---|
| Components | A | B | C | D |
| Ivermectin | 0.20 | 0.40 | 0.60 | 0.80 |
| Ethanol anhydrous | 5.8 | 8.5 | 11.0 | 13.5 |
| Isobutane | 23.9 | 21.9 | 20.0 | 18.2 |
| Shot mass | 29.9 | 30.8 | 31.6 | 32.5 |

Ivermectin was dissolved in ethanol anhydrous. This solution was filled into cans, a 50-μL metering valve was crimped onto the cans and isobutane was added under pressure through the valve. Actuators for oral inhalation are used with an orifice diameter of 0.25 mm.

### Example 8

Compositions depicted in the following table, packaged in aluminum alloy cans, fitted with 100-μL valves and oral actuators:

| | Mass of components per 100-μL shot (mg) | | | |
|---|---|---|---|---|
| Components | A | B | C | D |
| Ivermectin | 0.40 | 0.80 | 1.20 | 1.60 |
| Oleic acid | 0.1 | 0.1 | 0.1 | 0.1 |
| Ethanol anhydrous | 11.6 | 17.0 | 22.0 | 27.0 |
| Norflurane (HFA 134a) | 104.1 | 95.3 | 87.1 | 79.1 |
| Shot mass | 116.2 | 113.2 | 110.4 | 107.8 |

To manufacture these formulations oleic acid and ivermectin were dissolved in the corresponding amount of ethanol anhydrous and filled into aluminum alloy cut edge cans. Next, 100-μL metering valves were crimped onto them and Norflurane was added under pressure through the valve. Alternatively, oleic acid and ivermectin are dissolved in ethanol anhydrous, Norflurane is added into a pressurized mixing vessel and the formulation is filled into the cans through the valves already crimped onto them. Nasal actuators are used to apply the formulation in the nasal cavity. Oral actuators are used to apply the formulation to the oral cavity and to inhale into the lower airways.

### REFERENCES

1. Yang et al., Cellular & Molecular Immunology; doi.org/10.1038/s41423-020-0407-x
2. Caly L. et al., Antiviral Research 178 (2020) 104787.
3. United States Pharmacopoeia, monograph “Ivermectin”
4. European Pharmacopoeia, monograph “Ivermectin”
5. Mastrangelo E. et al., J Antimicrob Chemother 2012; 67: 1884-1894.
6. Crump A. The Journal of Antibiotics (2017), 1-11.
7. Guzzo C. A. et al., Journal of Clinical Pharmacology, 2002; 42:1122-1133.
8. Baraka O. Z. et al., Eur J Clin Pharmacol (1996) 50: 407-410.
9. Schulz J. D. et al., J Antimicrob Chemother 2019; 74: 1642-1647.
10. Schulz J. D. et al., Anal. Methods, 2018, 10, 2901.
11. Thomas L. (2020), retrieved from news-medical.net/news/20200427/Ivermectin-alone-not-useful-in-treating-COVID-19.aspx on May 15, 2020.
12. Lifschitz A. et al., Veterinary Parasitology 87 (2000) 327-338.
13. Bitter C. et al., Curr Probl Dermatol. Basel, Karger, 2011, vol 40, pp 20-35.
14. Garcia G. J. M. et al., Inhalation Toxicology, 2009; 21(7): 607-618.
15. Gizurarson S., Current Drug Delivery, 2012, 9, 566-582.
16. Pires A. et al., J Pharm Pharmaceut Sci, 12(3) 288-311, 2009.
17. Helassa N. et al., Biochem. J. (2014) 464, 213-220.
18. Wagenmann M. et al., Allergy Clin Immunol. 1992, 90(3) 419-423.
19. Humphrey et al., J Prosthet Dent 2001; 85:162-9
20. Chaccour C. el al., Nature Research, 2020, https://doi.org/10.1038/s41598-020-74084-y
21. Errecalde J. et al., Journal of Pharmaceutical Sciences, 2021, doi.org/10.1016/j.xphs.2021.01.017
22. Fr\check{o}hlich E. et al., Front. Pharmacol. 2016; 7:181.
23. Chrystyn H., Br J Clin Pharmacol, 2000; 51, 289-299.
24. De Backer W. et al., J. Aerosol Med. Pulmonary Drug Delivery, Vol. 23, No. 3, 2010, Pp. 137-148.
25. Therapeutic Goods Administration, Australian Public Assessment Report for Ivermectin, 2015
26. Starkloff W. J. et al., Pharmaceutical Development and Technology, 2016, dx.doi.org/10.1080/10837450.2016.1200078
27. Astier A. et al., FR3042412 A1 (French patent application, 2015)
28. Ali M. et al., Int. J. Drug Dev. & Res., April-June 2011, 3 (2): 240-247.

### Other Embodiments

While the invention has been described in conjunction with the detailed description thereof the foregoing description is intended to illustrate and not limit the scope of the invention. which is defined by the scope of the appended claims. Other aspects. advantages. and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references. documents. manuscripts and scientific literature cited herein are hereby incorporated by reference.

While this invention has been particularly shown and described with references to preferred embodiments thereof. It will be understood by those skilled in the art that various changes in form and details may be made therein without departing from the scope of the invention encompassed by the appended claims.

What is claimed:

1. A formulation comprising ivermectin in concentrations 0.1-5% m/V in a mixture of:
an alcohol from the group ethanol, isopropanol in a concentration of 1-50% m/m;
a propellant comprising: Norflurane, Apaflurane, propane, n-butane, isobutane, isopentane, n-pentane, heptafluoropropane or combinations thereof.

2. The formulation of claim 1, wherein the formulation is comprised within a delivery device fitted with a metering valve comprising a metered volume of between 10-200 μL and an oral actuator having an orifice diameter between 0.2 and 1 mm capable of delivering suitable doses of the formulation into the lower airways by inhalation or the oropharyngeal cavity.

3. A formulation comprising ivermectin in concentrations 0.1-2% m/V in a mixture of:
an alcohol in a concentration of 1-50% m/m;
a propellant selected from the group: Norflurane, Apaflurane, propane, n-butane, isobutane, isopentane, n-pentane, heptafluoropropane and combinations thereof.

4. The formulation of claim 3, wherein the formulation is comprised within a delivery device fitted with a metering valve comprising a metered volume of between 10-200 μL and a suitable nasal actuator capable of delivering suitable doses of the drug into the nose.

5. The formulation of claim 3, wherein the formulation is comprised within a delivery device fitted with a metering valve comprising a metered volume of between 10-200 μL and a suitable oral actuator capable of delivering suitable doses of the drug into the lower airways by inhalation or the oropharyngeal cavity.

6. The formulation of claim 1, where the propellant is Norflurane (HFA 134a).

7. The formulation of claim 3, where the propellant is Norflurane (HFA 134a).

8. The formulation of claim 1, further comprising excipients from the group tartaric acid, citric acid, hydrochloric acid, oleic acid, sorbitan trioleate, lecithin, povidone, polyethylene glycol 200 to 1000.

9. The formulation of claim 1, further comprising excipients selected from the group butylated hydroxytoluene and butylated hydroxyanisole.

10. A formulation comprising ivermectin, at least one alcohol, at least one propellant or combinations thereof.

11. The formulation of claim 10, comprising ivermectin in concentrations of about 0.1 to about 5% m/V.

12. The formulation of claim 10, wherein the at least one alcohol comprises a concentration of about 1 to about 50% m/m.

13. The formulation of claim 10, wherein the at least one propellant comprises Norflurane, Apaflurane, propane, n-butane, isobutane, isopentane, n-pentane, heptafluoropropane or or combinations thereof.

14. The formulation of claim 10, wherein the propellant is HFC 227 or (1,1,1,2,3,3,3heptafluoropropane) or HFA 134a (1,1,1,2-tetrafluoroethane).
